(19) United States
(12) Patent Application Publication
(10) Pub. No.: US 2018/0318011 A1
(43) Pub. Date: Nov. 8, 2018

(54) AUTOMATED GENERATION OF BONE TREATMENT MEANS

(71) Applicant: Karl Leibinger Medizintechnik GmbH & Co. KG, Mühlheim (DE)

(72) Inventors: Christian LEIBINGER, Mühlheim (DE); Wolfgang HÖLLER, Perg (AT); Michael MARTIN, Kirchzarten (DE)

(73) Assignee: Karl Leibinger Medizintechnik GmbH & Co. KG, Mühlheim (DE)

(21) Appl. No.: 15/773,126

(22) PCT Filed: Oct. 27, 2016
(86) PCT No.: PCT/EP2016/075923

§ 371 Date: May 2, 2018

(30) Foreign Application Priority Data
Oct. 27, 2015 (DE) 10 2015 118 318.8

(51) Int. Cl. A61B 34/10; A61F 2/28; G05B 19/4099

(52) U.S. Cl. CPC A61B 34/10; A61F 2002/30948; G05B 19/4099; A61F 2/2875

(57) ABSTRACT

The invention relates to a method for producing bone treatment means, with a first step in which original 3D data of a bone or of a bone portion of a specific patient to be treated are made available, wherein a site to be treated is present inside the bone or the bone portion, with a second step involving the use of 3D data of a reference patient who has been selected according to predefined criteria, wherein the 3D data correspond to the bone or to the bone portion with the site to be treated, and with a third and reconstructive step for supplementing or completing 3D data for the reconstruction of the site to be treated, wherein a mirroring step is used in which 3D data of the specific patient to be treated, which have their origin on a mirror-symmetrical other side of the patient, are superposed, specifically at a site corresponding to the bone or bone portion, in order to obtain the combined 3D data.

The invention relates to a method for producing bone treatment means, for example orthognathic osteosyntheses and/or templates.

Related methods are known, for example, from the U.S. Pat. No. 8,855,389 B1 and US 2014/0094924 A1. U.S. Pat. No. 8,855,389 B1 discloses a computer-implemented method for employing a finite-element technique for bone implant systems. In this context, also a library including pre-constructed implant data is accessed. Said data are applied/morphed onto an intact bone, however. In US 2014/0094924 A1, on the other hand, a mirror image of an intact/undamaged contra-lateral bone is made use of. Further state of the art is known from US 2010/0 151 400 A1.

In the existing methods for producing bone treatment means, viz. either implants or bone resection/bone cut templates, the quality is not satisfying. In this respect, an improvement is to be provided. Furthermore, bone treatment means adapted to the individual patients are to be enabled to be produced and made available more quickly, more inexpensively and more easily. Also, higher planning reliability is to be ensured. Planning is further intended to be facilitated. Finally, also the user friendliness of such method is to be enhanced.

In a method for producing bone treatment means this object is achieved by using different steps. In a first step, for example, (original) 3D data of a bone or a bone portion of a specific patient to be treated are to be detected/retrieved/utilized, wherein a site to be treated is present inside the bone or the bone portion. Said site usually is a defect or a bone material defect. Said (original) 3D data of a bone or a bone portion of the specific patient to be treated are based, for instance, on a data collection step, e.g. by means of CT, MRT, MRI, DICOM or similar methods and apparatuses. In a third reconstructive step, the two sets of data are linked to each other so that 3D data are supplemented or completed for the reconstruction of the site to be treated. Accordingly, trimming of the original 3D data or of the obtained supplemented data on the basis of the 3D data of the selected reference patient is or may be included, thus allowing to obtain reconstructed and trimmed 3 data for bridging or supplementing a bone material defect in the patient to be treated. This helps to achieve a substantial improvement as compared to previous methods. The data of the “reference patient” may especially relate to a data set which has been composed of different individual patients. Accordingly, for example formations of mean values, formations of medians and/or other/similar algorithms may be used. Hence the “reference patient” need not necessarily, but may be, understood to be an “individual person”. It suggests itself to compose an “artificial” “reference patient” from existing data sets. Finally, a statistic model is employed. A patient is meant to be a living or a dead person or animal and/or parts thereof. A mirroring step is used to obtain combined 3D data of the site to be treated by means of superposing 3D data of the specific patient to be treated onto the site concerned, wherein the superposed 3D data have their origin on a mirror-symmetrical other side of the patient, specifically at a site corresponding to the bone or bone portion. While already involving statistical data, i.e. the data which have been made available by one or more reference patients, shows an improvement, such improvement is significantly further improved by making use of a mirroring step and making use of the mirrored data of the sound side of the specific individual patient to be treated. Thus, also a step of involving 3D data is provided, wherein said 3D data correspond to the bone or the bone portion with the site to be treated (however on the sound side), and hence have their origin on a side that is mirror-symmetrical to the side to be treated.

Advantageous embodiments are claimed in the subclaims and shall be illustrated in detail in the following.

It is of advantage when the result of at least the three steps is used for planning the operation.

It is of further advantage when the three steps of making available the original 3D data of the patient to be treated, of involving the 3D data of the reference patient and of supplementing are run successively or at least partially in parallel. In this way, planning sections of 5 minutes to 10 minutes can be observed and even complete manufacture of ≤12 hours can be reached, when manufacture is carried out in situ, or of ≤48 hours, when a medical engineering enterprise is employed at a different location.

When after the third step the bone treatment means is produced in a fourth step in the form of an implant or an osteotomy template, a component to be fastened to the bone can be made available relatively quickly.

It has also turned out to be advantageous when in a preparation step the original 3D data of the patient and/or the 3D data of one or more reference patients are entered into an e.g. web-based data base and/or are gathered therefrom.

An advantageous embodiment is also characterized in that prior to the mirroring step and/or after the first step a computer-aided 2D or 3D visualization is performed. In this way, the user friendliness is increased.

In order to enable identification of the individual bones and, resp., bone fragments to be carried out efficiently and easily, it is of advantage when before or after the mirroring step, preferably after the step of visualizing, defined bone marker points will be/are selected, for example in the form of “landmarks” or markings. In order to be able to improve not only existing bones, but also to replace actually missing material it is of advantage when a bone material defect of the patient to be treated is closed or bridged or filled by type of a hole. In this way, the field of application of the method can be significantly broadened. Of course, it is also possible to utilize the bone treatment means so that, after being fastened to the bone/bone portion, it serves as a guiding and/or directing means for perforating, cutting and piercing the bone.

The patient can be provided with help more quickly than previously, when prior to the fourth step, i.e. manufacturing, in a generation step 3D data and/or manufacturing data for controlling production machines, e.g. NC or CNC data are generated and advantageously said NC or CNC data are directly or indirectly fed into a production device such as a control device of a milling, turning, sintering or welding system. Master-forming, reforming, especially machining and/or additive manufacturing methods then can be used quickly and efficiently. Especially advantageous is the use of rapid prototyping techniques such as 3D print techniques, especially those which make use of a *.3mf data format. Apart from geometrical information, also manufacturing information for additive and/or machining manufacture should be included.

It is of further advantage when preferably directly after the third step and/or prior to the fourth step a modelling step for attaining surfaces, axes, localizations and/or deviation factors is carried out.

It is useful when an operation planning step is carried out prior to the fourth step or instead of the fourth step.

An advantageous embodiment is also characterized in that the 3D data of the patient to be treated and/or the 3D data of the reference patient(s) are stored in/on a database of a hospital or in an I-cloud server (or a similar unit) or a database of a medical engineering enterprise. Both in-hospital, out-hospital and all-available data then can be used. Especially by a web-based solution the acceptance of the method is improved and the use is facilitated.

When the 3D data of the reference patient(s) contain selection criteria such as information about smoker/non-smoker, sex, age, size, profession, ethnics and/or constitution physiology, the selection of the respective (individually) matching data for reconstructing the bone is facilitated. Concerning the constitution physiology information, the classification according to Kretschmer is suited, although his classification is discussed in a controversial manner.

The invention also relates to an apparatus for carrying out a planning and/or manufacturing method, wherein means for carrying out the method according to the invention are contained/established and prepared.

A development consists in the fact that a computer is comprised/contained which is prepared and established for automatically carrying out the steps of the method. Thus, interaction with an operating staff is minimized.

Use according to the invention consists in inserting irregularities in a bone and thus obtaining a better diagnosis.

In other words, a method or process is described in which, while utilizing statistical form models, a surface and/or a volume is/are generated on and/or in which the implant reconstruction and the templates for osteotomy are deposited in a database. In this way, bone treatment steps and/or bone treatment means can be automatically adapted to and calculated for each individual. The bone treatment means can also be produced in individual adaptation and especially promptly.

Statistical models of anatomic regions are suitable for medical planning. These are virtual models that allow for supplementing or replacing missing or defective regions by way of existing individual form information.

It has turned out that the statistical models for reconstruction of the bone supporting apparatus of human beings enable/show higher accuracy than simple/singular mirroring of the sound side to the defective side. It is of great advantage that in automated reconstruction of the pathologically or traumatologically modified bone merely an orientation by way of points or surfaces on local bones is required for applying the statistical form model and for obtaining a reconstruction irrespective of more complicated segmentation methods. In addition, the type and quality of the present 3D image information of the individual now is independent of the result of reconstruction by the statistical model. This also means that the presence of artefacts, for example based on metal bolts, which cause blurred areas in imaging diagnosis methods can be segregated and thus can be removed.

When the statistical model is combined with implant constructions, this means that the latter can be adapted to the respective individual by an automated procedure. By selecting typical fracture localizations e.g. individual implants can be generated by an automated procedure in this way. It is a further idea to collect information of the individual reconstructions in order to thus obtain an implant optimization for standardized average implants.

The same principle can also be applied to the so-called “cutting guides”. “Cutting guides” are required for performing calculated osteotomies on the bone. For example, in a mandibular reconstruction in which a bone transplantation from the fibula is to be inserted, it is calculated in advance in which way the raised bone has to be cut so that the anatomic shape of the mandible can be reconstructed. When said defects are deposited in a database, the “cutting guides” can be calculated by an automated procedure. In addition, by such method the additional X-ray exposure of the donor region can be dropped in the future, when the statistical model is adapted to provide said information as an average value in an automated manner, which is assumed at present.

The process chain for manufacturing implants is as follows:
1. data collection (CT, MRT, ultrasound, statistic pattern (sex, age, size, profession . . . ))
2. selection of the region and/or of the implant by points or surfaces
3. application of a statistical model to the selected region
4. deformation of the implant to the assigned region
5. export of the finished implant construction file.

The process chain for the “cutting guide” can be characterized as follows:
1. data collection
2. selection of the region to be reconstructed
3. application of a statistical model to the selected region
4. selection of the donor region and calculation of the osteotomies required
5. representation of the required repositioning correction and automatic construction of the cutting guide
6. export of the finished construction file

Diverse advantages over other methods are resulting. For example, no mirroring of the side is necessarily used. In this way, the individual asymmetry can be taken into account. New construction of the implant is not required. Any number of “raw implants” can be deposited. They can be retrieved depending on the indication and the operating surgeon. “Cutting guides” can also be calculated in the operation planning method. The process chain is significantly reduced in this way. The required examination by the physician is dropped, as it is carried out in the same session of the implant generation by the planning person. The web-based application allows for quick and efficient planning without any additional software. The software can continuously improve the implants and the surfaces in the self-learning mode. It becomes possible to deposit “standard measures” and “standard axes” in order to detect pathological changes and to suggest the appropriate correction. An additional radiograph and a related radiation exposure of the donor region may be dropped.

Hence it is the special feature that an automatic reconstruction of the bone surface by 3D data takes place, specifically using present data of the specific individual patient that are supplemented by data from a statistical model. The combination of the present (residual) data of the individual patient with the supplementary 3D data from the statistical model therefore results in a pinpoint surface reconstruction of the bone to be treated.

The statistical form model serves for computer-aided planning. The shape model is integrated in the respective planning software (e.g. as STL data set) and may be used for “virtual reconstruction” in surgical navigation. It is the advantage of this method that mirroring need not, but can, be carried out for reconstruction. In this way, bilateral (two-sided) defects can be navigated. The simultaneous entraining of the virtual implants permits precise control of the surgical positioning by navigation.

Furthermore, a special application consists in the fact that a standardized implant is already “constructed” for a region. I.e. an “average implant” was already generated by way of standard mean values. Said average implant is deposited in a database. By way of the construction points, it is anchored in the statistical model and is automatically placed at the appropriate site of the individual. In a second step, the surface of the implant area facing the bone then is adapted. The construction file varies when the statistical form model is adapted to the individual bone.

It is also a special feature, when a standard implant is supported on the appropriate site of the bone (best fit). By a trimming method material is filled between the surface of the implant facing the bone and the bone.

Hereinafter the invention shall be illustrated in detail by way of several Figures, wherein:

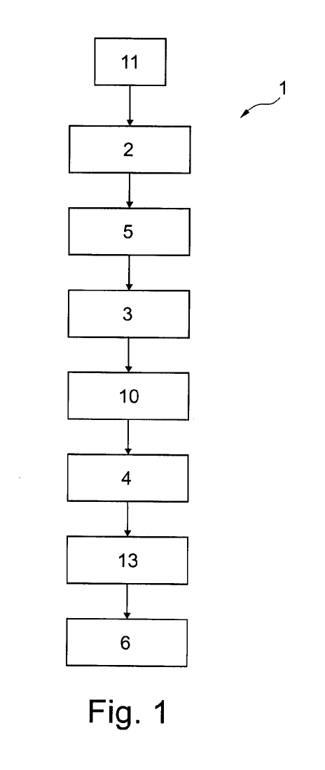

FIG. 1 shows a flow chart for carrying out a method according to the invention,

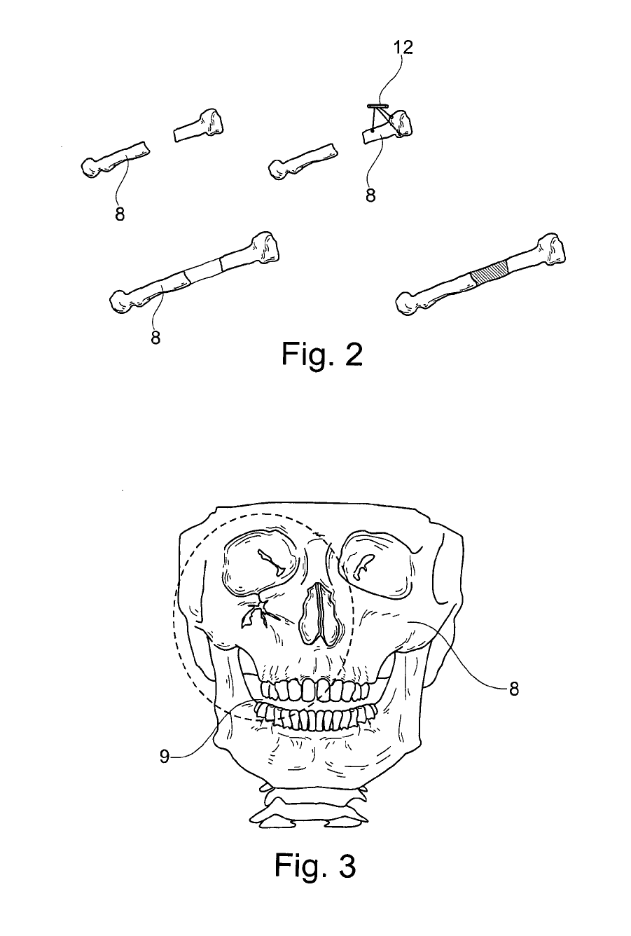

FIG. 2 shows the course of remodeling on a bone,

FIG. 3 shows the position of an area to be treated on an exemplary skull and

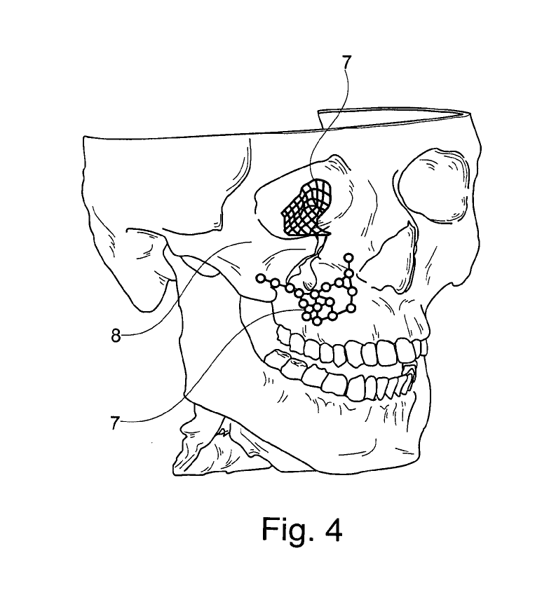

FIG. 4 shows the mounting of bone fastening means, by type of an eye socket implant and a maxilla implant.

The Figures are merely schematic and only serve for the comprehension of the invention. Like elements are provided with like reference numerals.

The invention is appropriate for use in the skull and face surgery, but it may finally be used on and/or for each osseous structure of a human being or a mammal.

In a method 1 according to the invention, there is a first step 2 of making available original 3D data of a bone or a bone portion of a specific patient to be treated. This is followed by a second step 3 in which involving of 3D data of a reference patient who has been selected according to predefined criteria takes place, namely the 3D data are gathered in a comparable region which is due to be treated. In a following third step 4 supplementing, possibly comprising trimming, of the 3D data combined of step 2 and step 3 is performed, wherein combining of the data takes place in a partial step.

Between the first step 2 and the second step 3 also a mirroring step 5 may take place. In said mirroring step, 3D data which are opposed to the longitudinal axis or a plane of symmetry including the longitudinal axis of the body are gathered from a sound site on the ill (specific) patient to be treated and are superposed to the 3D data of the ill side to be treated. It is recommendable to make use of this step.

In a fourth step 6, also referred to as manufacturing step, a bone treatment means 7 is manufactured for example by type of an implant or an osteotomy template. Thus also “virtual surgical planning” is possible. Such bone treatment means 7 which is fastened to a bone 8 of a specific individual patient to be treated is shown in FIG. 4. FIG. 3 illustrates an area 9 to be treated on a skull including a bone 8. While the eye socket of said skull has a defect in the area of the region 9 to be treated on the right side when viewed from the patient, the eye socket has no defect on the left side when viewed from the patient.

The respective data of the sound side are transmitted to the defective site in a mirroring step 10 visualized in FIG. 1. They are morphed thereon/therein. Preceding the previous steps, there is a preparation step 11 in which the 3D data of the patient and/or the 3D data of one or more reference patients are entered into a local or web-based database and, resp., are gathered therefrom.

FIG. 2 depicts in which way, starting from a bone defect, landmarks are created, then an “adjustment” takes place in which a superposed form model is used which is not yet adapted to subsequently insert a statistical form model in a calculation cut so as to obtain an adapted model with a replaced bone defect. Markers 12 that form the “landmarks” are characterized by the reference numeral 12.

Hence, the point is that so far exclusively e.g. skull defects have been reconstructed in most cases by mirroring of the sound side to the defective side. This is only matching to a limited extent, however, or the results are not sufficient. In the present method, a plurality of skull models is evaluated to form a statistical model. From the statistical model the defective site now can be reconstructed on the defective skull.

In the method 1 according to the invention a generation step 13 is used.

### REFERENCE NUMERALS

1 method
2 first step (data for making available)
3 second step (involving a statistical model)
4 third step (supplementing plus trimming, where appropriate)
5 mirroring step
6 fourth step/manufacturing step
7 bone treatment means
8 bone
9 region to be treated
10 mirroring step
11 preparation step
12 marker
13 generation step

1. A method for producing bone treatment means comprising:
a first step in which original 3D data of a bone or a bone portion of a specific patient to be treated are provided, wherein a site to be treated is present inside the bone or the bone portion;
a second step of involving 3D data of a reference patient who has been selected according to predefined criteria, wherein the involved 3D data of the reference patient correspond to the bone or the bone portion with the site to be treated and are composed of 3D data of various individual patients by means of formatting a mean value;
a reconstructive step for supplementing or completing the 3D data combined of the first step and the second step for the reconstruction of the site to be treated.

2-9. (canceled)

10. An apparatus for carrying out a planning and/or manufacturing method, wherein means are contained and prepared for carrying out the method (4-) according to claim 1.

11. The method according to claim 1, further comprising:
a mirroring step in which 3D data of the specific patient to be treated which have their origin on a mirror-symmetrical other side of the patient are superposed, specifically at a site corresponding to the bone or bone portion, in order to obtain the combined 3D data.

12. The method according to claim 1,
wherein the first step, the second step, and the reconstructive step are run successively or in parallel.

13. The method according to claim 11,
wherein the first step, the second step, the reconstructive step, and the mirroring step are run successively or in parallel.

14. The method according to claim 1, further comprising:
after the reconstructive step, a step of producing the bone treatment means in the form of an implant or an osteotomy template.

15. The method according to claim 14, further comprising:
a preparation step in which the original 3D data of the patient and/or the 3D data of one or more reference patients are entered into a database and/or are gathered therefrom.

16. The method according to claim 11, further comprising:
before the mirroring step and/or after the first step, performing a computer-aided 2D or 3D visualization.

17. The method according to claim 11, further comprising:
before or after the mirroring step, selecting defined bone marker points.

18. The method according to claim 1,
wherein a bone material defect of the patient to be treated by type of a hole is closed or bridged or filled.

19. The method according to claim 14, further comprising:
prior to the producing step, generating 3D data and/or manufacturing data for controlling manufacturing machines.

20. The method according to claim 1,
wherein the result of at least the first step, the second step, and the reconstructive step are used for planning the operation.
